(19) United States
(12) Patent Application Publication
(10) Pub. No.: US 2017/0052182 A1
(43) Pub. Date: Feb. 23, 2017

(54) OPTOELECTRONIC CARTRIDGE FOR CANCER BIOMARKER DETECTION UTILIZING SILICON NANOWIRE ARRAYS

(71) Applicants: University of Iowa Research Foundation, Iowa City, IA (US); Advanced Silicon Group, Inc., Lincoln, MA (US)

(72) Inventors: Marcie R. Black, Lincoln, MA (US); Fatima Toor, Coralville, IA (US); Aliasger Salem, Coralville, IA (US)

(21) Appl. No.: 15/243,099

(22) Filed: Aug. 22, 2016

Related U.S. Application Data
(60) Provisional application No. 62/208,536, filed on Aug. 21, 2015.

(51) Int. Cl. G01N 33/574; G01N 21/31; G01N 33/552; G01N 27/02; H01L 29/16; H01L 29/06

(52) U.S. Cl. CPC G01N 33/574; H01L 29/16; H01L 29/0669; G01N 33/552; G01N 27/02; G01N 21/31

(57) ABSTRACT

Provided is a biosensor including a nanowire array. According to an example, the nanowire may include at least 1000 nanowires per cm², the at least 1000 nanowires per cm² including individual nanowires each defined by a longitudinal surface and a vertical surface, the longitudinal surface being at least two times longer than the vertical surface, where the vertical surfaces of each of the individual nanowires is configured to couple to a substrate.

### RELATED APPLICATIONS

This application claims priority under 35 U.S.C. §119(e) to U.S. Provisional Application Ser. No. 62/208,536, titled “OPTOELECTRONIC CARTRIDGE FOR CANCER BIOMARKER DETECTION UTILIZING SILICON NANOWIRE ARRAYS,” filed on Aug. 21, 2015, which is hereby incorporated herein by reference in its entirety.

### BACKGROUND OF THE INVENTION

According to certain sources, millions of people are diagnosed with cancer, or related illness, each year. Unfortunately for many of the diagnosed, the survival rates are not optimistic. Accordingly, billions of dollars in resources and capital are being expended each year to care for and provide health care services to those diagnosed with cancer related illnesses. In particular, the Agency for Healthcare Research and Quality (AHRQ) estimated that the sum of all health care costs for cancer in the United States in 2011 was approximately $88.7 billion.

Often, targeted therapy can be useful in targeting particular types of cancer. Targeted therapy typically includes application of specialized drugs to fight the responsive type of cancer. Targeted therapies are typically useful when a corresponding mutation is identified, and tend to provide the benefits of improve efficacy with less side effects, when compared to generic cancer treatments.

Early detection can also lower the cost of treatment, and increase the survival chances of a diagnosed patient. According to some estimates, treating a patient for cancer can averages around a million dollars per patient. Various costs can be distributed among costs associated with hospital outpatient or doctor office visits, inpatient hospital stays, and prescription drugs, with the majority of expenses being associated with the outpatient or doctor office visits.

While various early detection methods do exist, existing approaches have inconsistent levels of accuracy and efficacy. In particular, conventional cancer detection approaches are generally not suitable for early detection. Currently, cancer is first detected by physical examination in combination with imaging studies, such as CT scans or MRIs. Routine blood tests may also reflect organ dysfunction, or other symptoms which are caused by cancer. However, such approaches generally are most effective after the cancer has significantly damaged the patient, at which point it may be too late for treatment. Moreover, existing approaches are unable to detect all types of cancer cells.

Accordingly, a low-cost early detection method for cancer is not only critical to relieve the economic burdens on the healthcare system, but to also save lives.

### SUMMARY OF THE INVENTION

Aspects and embodiments are generally directed to biosensors that are made using arrays of vertical silicon nanowires which offer an increased surface area when compared to typical horizontally arranged arrays. In certain examples, the nanowires are incorporated onto a test chip of the biosensor, which may be exposed to a sample and to determine the presence, or absence, of a biomarker within the sample. In some instances, the amount of the biomarker can be quantified using the biosensor, for example ng/ml or strands of DNA/ml.

According to certain examples, the density nanowires within the nanowire array may be more than 100, 1000, 100,000, or 1,000,000 nanowires per cm². Various examples of the nanowire array discussed herein have a unique device design which eliminates the challenge of contacting individual horizontal nanowires, thus allowing nanowire arrays with a higher density of individual nanowires.

In certain examples, the discussed design allows the biosensor to be used to measure both optical and electrical signals individually, or simultaneously. By offering two different types of measurements via the same test chip, the biosensors offer the potential of decreased false positives.

In some examples, the biosensors can be used to detect a variety of biologically produced molecules with the same test chip, where the test chip can be a disposable silicon nanowire chip which measures the biologically produced molecules electrically and/or optically. Each of the individual nanowires can be functionalized (e.g., coated with a desired chemical) to include binding agents which are capable of selectively binding to molecules of interest within the test sample. For example, the nanowires can be functionalized to include binding agents that selectively bind to biologically produced molecules that are used to diagnosis various physiological states, such as diabetes, high cholesterol, and the like, as well as various other biologically produced molecules associated with diseases, such as specific types of cancer DNA. In addition, the biosensor can be used to test multiple biomarkers with the same test chip, such as DNA and proteins.

The design of the discussed nanowire-based biosensor, and in particular, the nanowire array, allows the nanowires to be electrically contacted through a base of the nanowire array. In particular, the individual nanowires of the array may be attached to a substrate through the base of the nanowire array, and not at a front surface, where the nanowires are exposed to the sample. According to various examples, a sample applied to the test chip can be a liquid or gas sample, such as blood, urine, or sweat. According to certain examples, the individual nanowires can be made from silicon including single crystalline silicon, polysilicon silicon, or amorphous silicon, among other suitable materials.

In certain examples, at least one p-n junction may be formed between the base of the nanowire array and the substrate. The p-n junction can be made by standard silicon processing techniques, such as diffusion of dopants. For example, a process of producing the p-n junction may include: providing a p-type wafer, forming the nanowire array, and doping the individual nanowires and base as an n-type material, such that the p-n junction forms below the nanowire array.

As mentioned herein, the nanowires can be functionalized (e.g., coated with a chemical) to include binding agents such as receptors, antibodies, and/or nucleic acid sequences that specifically bind to biomarkers. In some instances, a single device or test chip can include multiple subarrays of functionalized nanowires (e.g., groups or “subarrays”). Each subarray may then be functionalized to detect a specific biomarker. In certain examples, a sample applied to the biosensor can either be divided within the test chip and individually exposed to the individually functionalized nanowire subarrays, or in some instances, passed over the functionalized nanowire arrays in series such that the same sample sees more than one subarray.

Given the benefit of this disclosure, one of ordinary skill in the art will appreciate that when the individually functionalized nanowire subarrays are in series, the order and placement of the subarrays in the series will need to be selected such that the biomarkers of interest flow through to the appropriate functionalized nanowire subarray.

According to certain examples, the subarrays may be electrically isolated from each other and individually electrically accessible. The electrical access to the subarrays may include an array similar to those used for memory or LEDs within a display. In certain examples, the subarrays can be electrically isolated with silicon dioxide using standard semiconductor processing technologies, such as trench and field isolation.

According to various examples, electrical measurements performed by the biosensor may be executed using a current-voltage measurement of the nanowire array. For example, the current-voltage measurement may be performed with the presence of incident light, or with a difference in the current-voltage measurement and with and without the incident light. In certain examples, the light intensity may be modified by the biosensor.

In addition, in some implementations the wavelength of the incident light may be varied. According to some examples, the incident light can be monochromatic, with one dominant wavelength of light. For example, wavelength of light can be scanned throughout a range of wavelengths, or example from 350 nm to 700 nm.

In addition to electrical measurements and opto-electrical measurements, in some examples the nanowire-based bio sensor can also be used to perform purely optical measurements on biomarkers. Since the nanowires have a high density (e.g., 1,000 nanowires per cm²), and may capture specific biomarkers, an optical measurement may alone be sufficient to detect the presence of biomarkers in some situations. For example, the optical spectra of the biomarkers can be used to determine the biomarker presence. Measuring the presence of the biomarkers using multiple methods (optical and electrical) will likely decrease the detection of false positives. Optical measurements may include reflection, scattering, and transmission. Transmission through the sample may be possible for certain wavelengths of light not absorbed in the silicon substrate, for example, in the infrared (IR) spectrum at sub-bandgap radiation.

Still other aspects, embodiments, and advantages of these exemplary aspects and embodiments are discussed in detail below. Embodiments disclosed herein may be combined with other embodiments in any manner consistent with at least one of the principles disclosed herein, and references to “an embodiment,” “some embodiments,” “an alternate embodiment,” “various embodiments,” “one embodiment” or the like are not necessarily mutually exclusive and are intended to indicate that a particular feature, structure, or characteristic described may be included in at least one embodiment. The appearances of such terms herein are not necessarily all referring to the same embodiment. Various aspects and embodiments described herein may include means for performing any of the described methods or functions.

### BRIEF DESCRIPTION OF THE DRAWINGS

Various aspects of at least one embodiment are discussed below with reference to the accompanying figures, which are not intended to be drawn to scale. The figures are included to provide illustration and a further understanding of the various aspects and embodiments, and are incorporated in and constitute a part of this specification, but are not intended as a definition of the limits of the invention. In the figures, each identical or nearly identical component that is illustrated in various figures is represented by a like numeral. For purposes of clarity, not every component may be labeled in every figure. In the figures:

FIG. 1 is a schematic illustration of silicon nanowire formation using a metal enhanced etching process;

FIG. 2A is a schematic illustration of an example of horizontal nanowire array fabrication;

FIG. 2B is a further schematic illustration of the horizontal nanowire array fabricated according to FIG. 2A;

FIG. 2C is a graph illustrating conductance data measured from two of the horizontal nanowire devices of FIG. 2B;

FIG. 2D is a graph illustrating complementary sensing using a p-type nanowire device of FIG. 2B and an n-type nanowire device of FIG. 2B;

FIG. 2E is an illustration of the horizontal nanowire array of FIG. 2B detecting multiple proteins;

FIG. 2F is a graph illustrating conductance data measured from three nanowire arrays fabricated according to FIG. 2A;

FIG. 3A is a process flow for fabricating a biosensor according to aspects of the invention;

FIG. 3B is a process flow for fabricating a biosensor according to conventional processes;

FIG. 4A is a graph illustrating a current-voltage curves for solar cells;

FIG. 4B is a graph illustrating a conductance-voltage curves for solar cells;

FIG. 5 is an example of a nanowire array according to aspects of the invention;

FIG. 6A is an example illustration of an optoelectronic sensor system, according to aspects of the invention;

FIG. 6B is a further illustration of the nanowire arrays illustrated in the system of FIG. 6A, according to aspects of the invention;

FIG. 6C is an illustration of the nanowire array having an EDC-NHS surface modification, according to aspects of the invention;

FIG. 6D is an illustration of antigen biomarkers introduced into the nanowire array, according to aspects of the invention;

FIG. 7 is a graph illustrating external quantum efficiency (EQE) curves for identical solar cells;

FIG. 8 is a graph illustrating the percent change in the optically induced current in a nanowire array after exposure to PSA compared to the optically induced current before exposure, according to aspects of the invention;

FIG. 9 is a schematic diagram of an example test chip, according to aspects of the invention;

FIG. 10 is an enhanced view of the example test chip of FIG. 9, according to aspects of the invention;

FIG. 11. is a block diagram of an example nanowire-based biosensor according to aspects of the invention;

FIG. 12 is a schematic illustration of another example of a test chip, according to aspects of the invention; and

FIG. 13 is a schematic diagram of a test chip including a nanowire array, according to certain aspects of the invention.

### DETAILED DESCRIPTION OF THE INVENTION

Before describing the particular aspects and embodiments of the present invention in detail, it is to be understood that this invention is not limited to specific solvents, materials, or device structures, as discussed with respect to particular aspects, embodiments, and examples as such may vary. It is also to be understood that the terminology used herein is for the purpose of describing particular embodiments only, and is not intended to be limiting.

Examples of the systems and methods discussed herein are not limited in application to the details of construction and the arrangement of components set forth in the following description or illustrated in the accompanying drawings. The systems and methods are capable of implementation in other embodiments and of being practiced or of being carried out in various ways. Examples of specific implementations are provided herein for illustrative purposes only and are not intended to be limiting. In particular, acts, components, elements and features discussed in connection with any one or more examples are not intended to be excluded from a similar role in any other examples.

Also, the phraseology and terminology used herein is for the purpose of description and should not be regarded as limiting. Any references to examples, embodiments, components, elements or acts of the systems and methods herein referred to in the singular may also embrace embodiments including a plurality, and any references in plural to any embodiment, component, element or act herein may also embrace embodiments including only a singularity. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. In addition, in the event of inconsistent usages of terms between this document and documents incorporated herein by reference, the term usage in the incorporated features is supplementary to that of this document; for irreconcilable differences, the term usage in this document controls.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also disclosed, as well as the range of values greater than or equal to 1 μm and the range of values less than or equal to 8 μm.

Aspects and embodiments are generally directed to sensors (e.g., biosensors) including large arrays of nanowires. For example, the array of nanowires may be formed on a test chip. The sensor of various embodiments may be constructed by fabricating at least one nanowire array, forming a solar cell by doping a top surface of a substrate, electrically contacting the substrate to the nanowire array, and functionalizing (e.g., chemically coating) the nanowires. As discussed, in certain examples the nanowires are incorporated onto a test chip which may be exposed to a sample to determine the presence, or absence, of a biomarker within the sample. In some instances, the amount of biomarker can be quantified using the biosensor.

Nanowire arrays are seeing increasing use in a variety of applications. U.S. Patent Application Publication No. 2009/256134 titled “PROCESS FOR FABRICATING NANOWIRE ARRAYS” offers one such example of a nanowire array, and is incorporated by reference herein in its entirety. A typical nanowire array might consist of a collection of silicon nanowires, on the order of 100 nm in diameter, and on the order of about a hundred nm to hundreds mm in height. Each nanowire may have an approximately cylindrical or frustoconical shape. In contrast to typical nanowire arrays which arrange each individual nanowire in a horizontal orientation relative to associated base surface, various examples of the nanowires discussed herein have an which may run approximately parallel to each other and in a vertical direction relative to the base surface (e.g., the substrate to which the nanowires are mounted). Accordingly, each nanowire may be attached at an end to the silicon substrate.

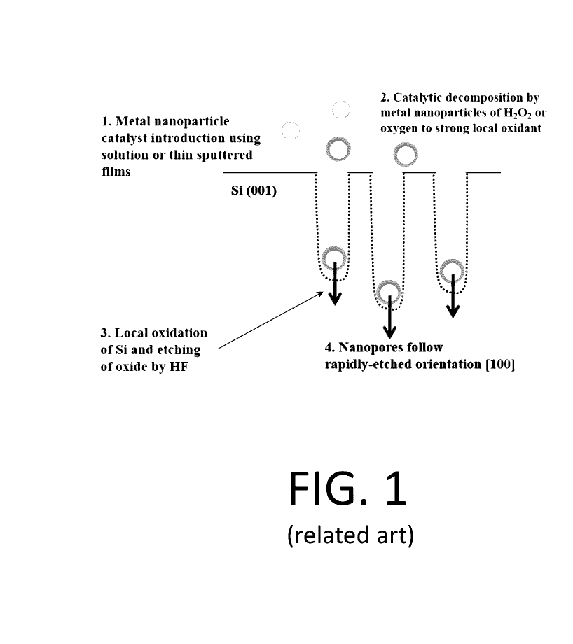

A common method for making silicon nanowires is metal-enhanced etching of a silicon-containing substrate. This process can be used to control the nanowire dimensions and is described in U.S. Pat. No. 8,143,143, titled “PROCESS FOR FABRICATING NANOWIRE ARRAYS”, and U.S. Pat. No. 8,450,599, titled “NANOSTRUCTURED DEVICES”, which are both incorporated by reference in their entirety. During metal-enhanced etching processes, a metal is deposited on a top surface of a silicon substrate and placed in a solution. While in the salutation, the etch is enhanced at the points where the silicon touches the metal. Since the metal coverage is not uniform, parts of the silicon are not etched leaving silicon with a graded index of refraction, cliffs, or nanowires. The metal used can be, for example, gold, platinum, or silver. FIG. 1 illustrates one example of silicon nanowire formation using a metal enhanced etching process according to a typical process.

Other typical techniques for forming silicon nanowires may include reactive ion etching and VLS (Vapor-Liquid-Solid). During VLS processes, nanowires are grown on a substrate using a metal catalyst and silane.

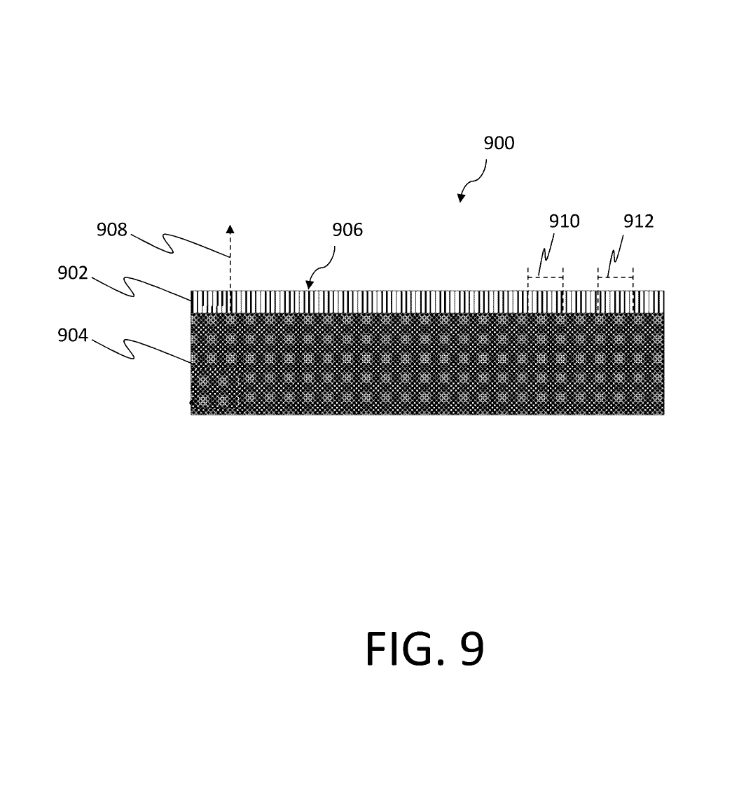

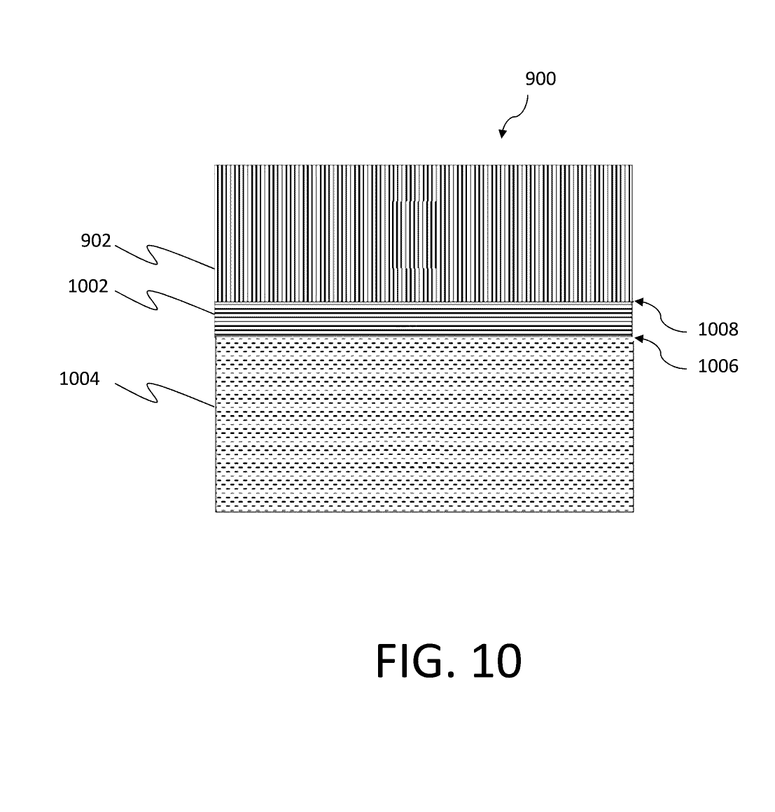

According to various aspects and embodiments, each nanowire of the discussed array has a high surface area to volume ratio, and therefore has the characteristics to make a very sensitive detector. As further discussed below with reference to at least FIGS. 9-13, in certain examples, each individual nanowire of the nanowire array may be defined by a longitudinal surface and a vertical surface. In certain examples, the longitudinal surface of each nanowire is at least two times longer than the vertical surface. Accordingly, the vertical arrangement of the nanowire allows the nanowire to have a significantly increased density of individual nanowires (e.g., at least 1000 nanowires per cm²) when compared to typical horizontal arrangements. Such an arrangement significantly improves the sensitivity of the biosensor.

As discussed herein, in various aspects and embodiments the biosensor may include an array of nanowires which are configured to measure indications of cancer, and other illnesses. Such aspects and embodiments offer the benefits of earlier cancer detection, and less medical waste (e.g., smaller blood samples). In particular embodiments, the plurality of nanowires may be constructed from silicon, which is a useful material because it is inexpensive and non-toxic.

According to certain aspects and embodiments, each individual nanowire of the array may be functionalized to detect a given biomarker (e.g., a cancer biomarker). As discussed herein, functionalization may refer to coating a nanowire with a desired chemical which is sensitive to a biomarker (e.g., a biomarker binding agent). When the functionalized nanowires are exposed to biomarkers, their electrical properties may change. Each nanowire may be constructed from silicon and may be highly sensitive to biomarkers once functionalized, at least because of the high surface area to volume ratio.

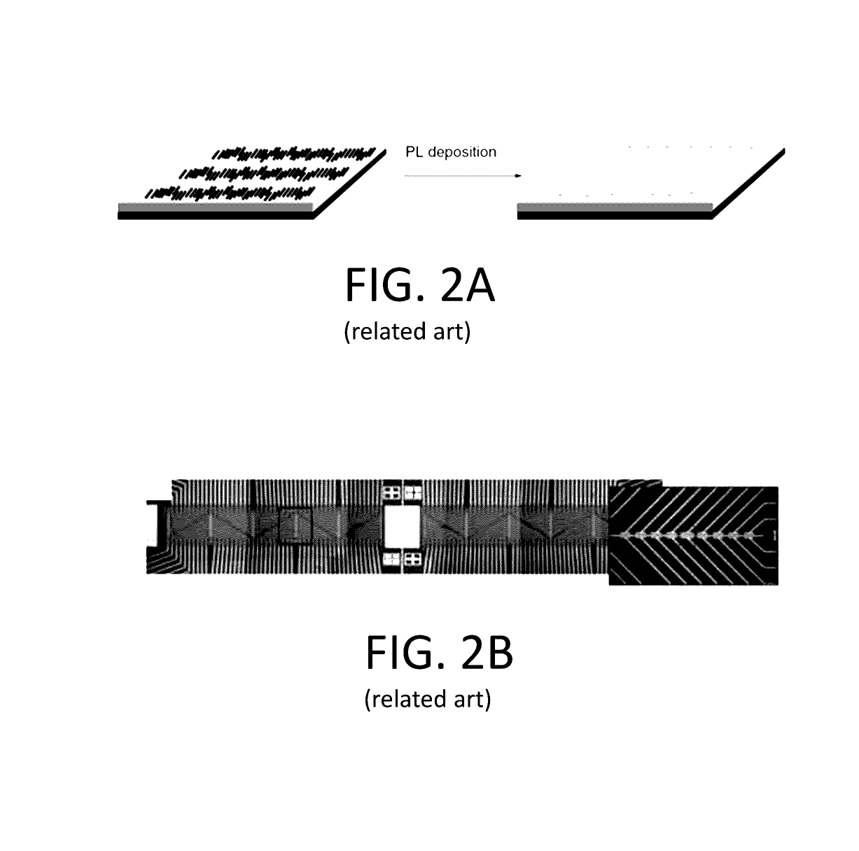

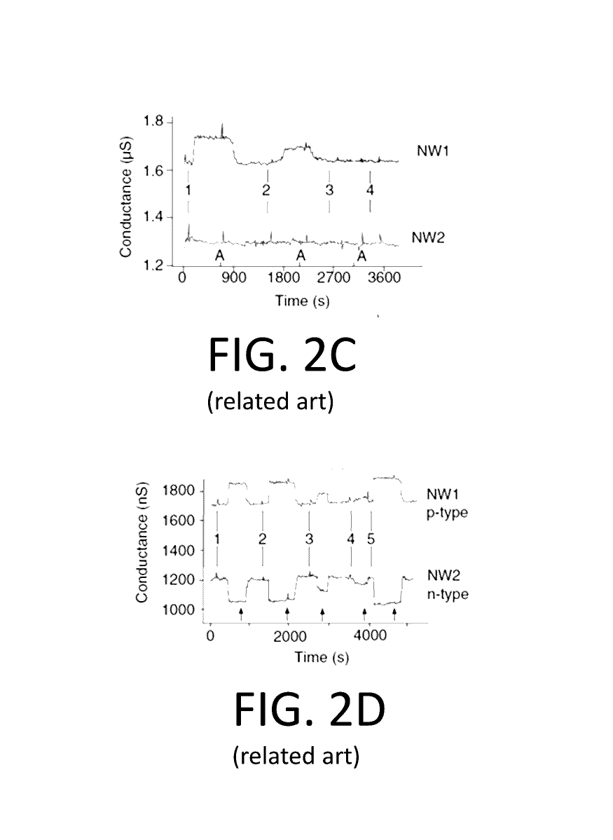

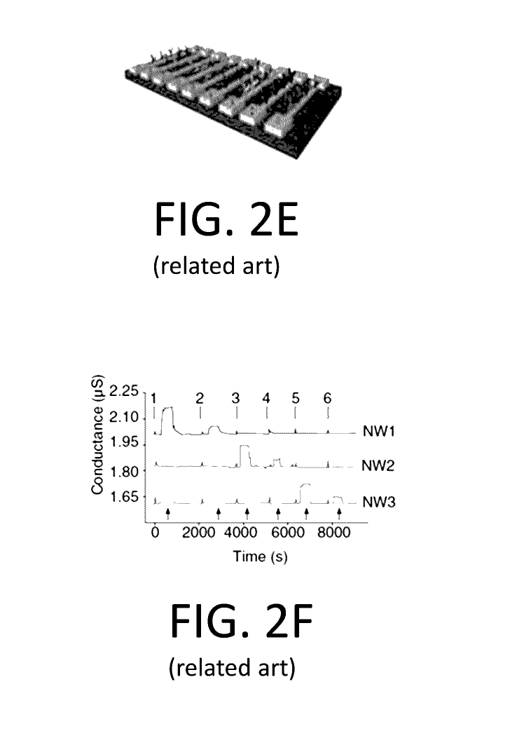

FIGS. 2A-2F provide an illustrative example of a typical horizontal nanowire array, and a process for forming the same, for the sake of comparison. In particular, FIG. 2A is a schematic illustration of an example of horizontal nanowire array fabrication. FIG. 2B is a further schematic illustration of the horizontal nanowire array fabricated according to FIG. 2A. FIG. 2C is a graph illustrating conductance data measured from two of the horizontal nanowire arrays of FIG. 2B. FIG. 2D is a graph illustrating complementary sensing using a p-type nanowire device of FIG. 2B and an n-type nanowire device of FIG. 2B. FIG. 2E is an illustration of the horizontal nanowire array of FIG. 2B detecting multiple proteins. FIG. 2F is a graph illustrating conductance data measured from three nanowire arrays fabricated according to FIG. 2A.

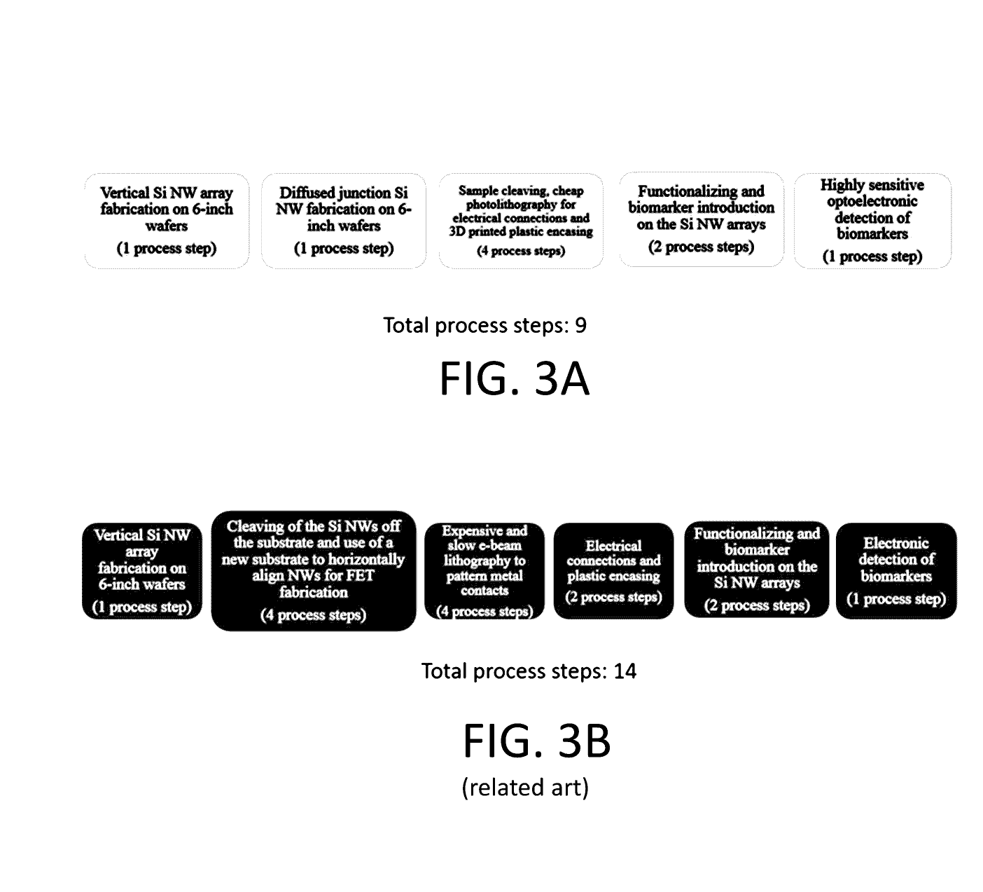

Some previous approaches to biomarker detection using nanowires have suggested the use of only electrical measurements, such as conductance probing. For example, in one such approach distinct nanowires and surface receptors are incorporated into horizontal nanowire field-effect-transistor arrays. However, electrical detection for horizontal wires requires that each of the nanowires is electrically contacted on both sides. This requirement makes using many nanowires challenging at least because of the complex device fabrication steps to contact the wires. FIGS. 3A and 3B offer a comparison of a process for fabricating a biosensor having a horizontal arrangement, and a process for having a vertical arrangement according to aspects discussed herein. In particular, FIG. 3A illustrates a process for fabricating a biosensor according to aspects and embodiments, and FIG. 3B illustrates an example process for fabricating a biosensor according to a typical horizontal approach.

Some other typical approaches for biomarker detection have included porous silicon sensors. For example, the change in a refractive index, photoluminescence spectra of fluorescence porous silicon has been used for the detection of biomarkers. In addition, changes in capacitance and conductance of the porous silicon was also used by electrically contacting the top of the porous silicon and having a second contact to electrically contact the bottom of the porous silicon layer.

Currently, some commercial sensors capable of real-time measurement of multiple biomarkers are available. At the core this technology are nanowires, microscopic wires whose conductance varies (with great sensitivity) as the concentration of target molecules passing over the nanowires change. However, these arrangements suffer the same shortcomings as those discussed above with reference to the horizontal arrangements.

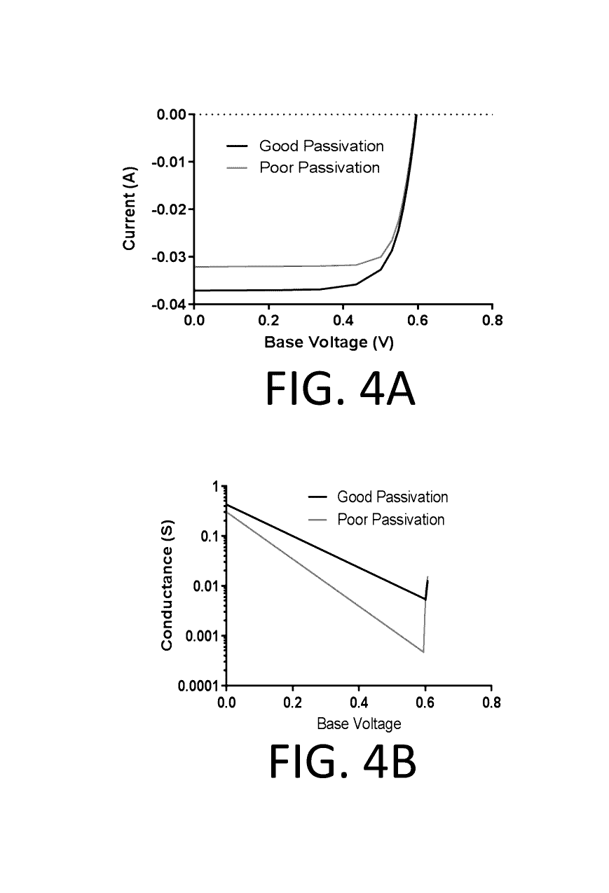

Often, nanowire arrays may be arranged as a solar cell. Charged particles effect the surface passivation on the surface of a solar cell. Accordingly, the quality of surface passivation affects the performance the solar cell. FIGS. 4A and 4B show a simulated current-voltage and conductance-voltage curves for solar cells that either exhibit good or bad surface passivation. The simulation data was generated by a commonly used solar cell simulator program, where the surface recombination velocity was changed to vary the surface passivation properties. Any defects or impurities at or within the surface of the semiconductor promote recombination. Since the surface of the solar cell represents a disruption of the crystal lattice, the surfaces of the solar cell are a site of particularly high recombination. The high recombination rate in the vicinity of a surface depletes this region of minority carriers.

Surface recombination velocity may be used to specify the recombination at a surface. In a surface with no recombination, the movement of carriers toward the surface is zero, and hence the surface recombination velocity is zero. In a surface with infinitely fast recombination, the movement of carriers toward this surface is limited by the maximum velocity they can attain. Accordingly, given the various approaches discussed herein, the vertical arrangement of nanowires in a corresponding array may also be used to provide an improved solar cell.

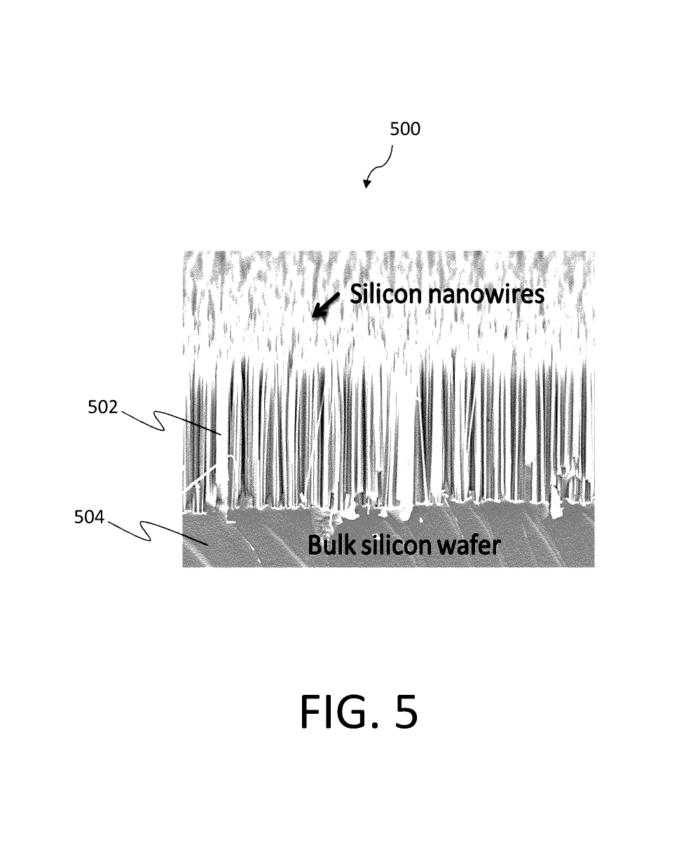

As discussed above, various aspects and embodiment are directed to a biosensor including an array of nanowires. In particular, aspects and embodiments may solve the challenges of efficiently contacting the nanowires to a mounting surface (and electrical contact) by using vertical silicon nanowires and a unique arrangement. The aspects and embodiments discussed herein allow measurements of approximately 1 billion nanowires per square centimeter. For instance, FIG. 5 illustrates one example of the density of individual nanowires 502 within an array 500, according to an example. FIG. 5 further illustrates a substrate 504 to which each nanowire 502 is coupled. In certain examples, the nanowire array 500 can be probed to determine the extent of cancer biomarkers conjugated on one or more surfaces (e.g., a front surface) of the array 500. This new method of detection enables sensors with nanowire arrays including many more individual nanowires than typical sensors, resulting in higher sensitivity device.

Various aspects and embodiments of the biosensor discussed herein are more sensitive than other detectors because the surface area of the biosensor. In particular, the surface area may be over a thousand times greater than that of a flat surface or a single nanowire device. Moreover, unlike other approaches that have used nanowires to detect biomarkers, the discussed approach is much easier to scale and manufacture since the manufacturer does not require electrical contact to each individual nanowire. Instead, one can look at the effect of the change in the electrical properties of the nanowires when exposed to biomarkers on the underlying test chip, thus allowing many more nanowires to be measured.

In certain examples, each individual nanowire (e.g., nanowires 502) of the plurality can be measured by probing the current-voltage with and without illumination. By looking at the changes in the electrical properties of the nanowire arrays the biosensor can electrically detect cancer markers without electrically contacting the top of the nanowires. In one example, without the attachment of the biomarkers, the nanowire surface will see a reduction in the level of electrical passivation. Accordingly, any carriers created at the surface of the nanowire array will recombine. By shining a light that is absorbed by the nanowire array, and sensing a change in the electrical signal with illumination, the biosensor can determine how many antigen biomarker molecules are attached to the nanowires. If no biomarkers are attached, the test chip will be unpassivated and there will be a minimal optical response. Otherwise, the test chip will be passivated and the photo-created carriers will be collected from the test chip leading to a strong optical response.

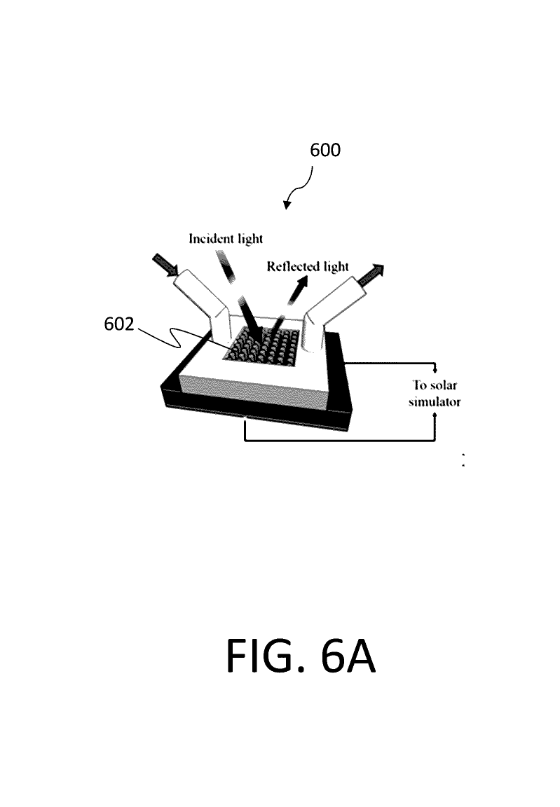

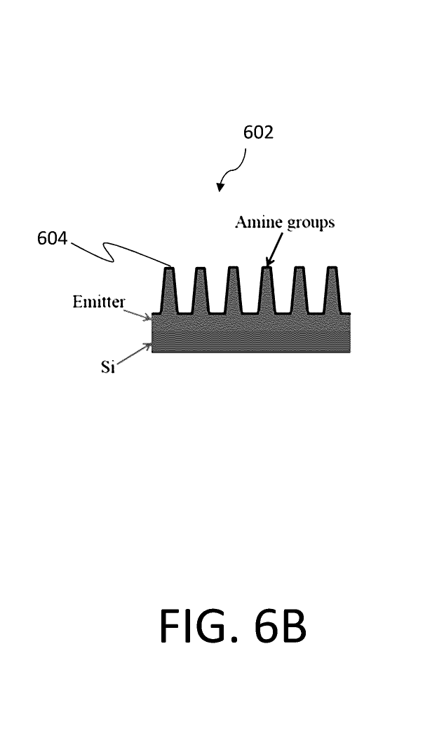

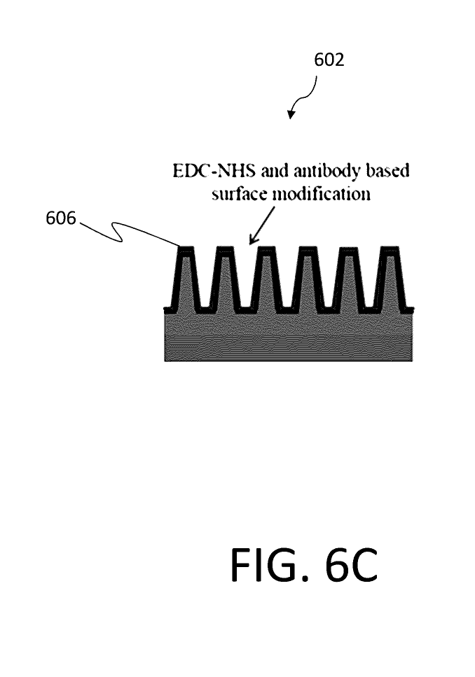

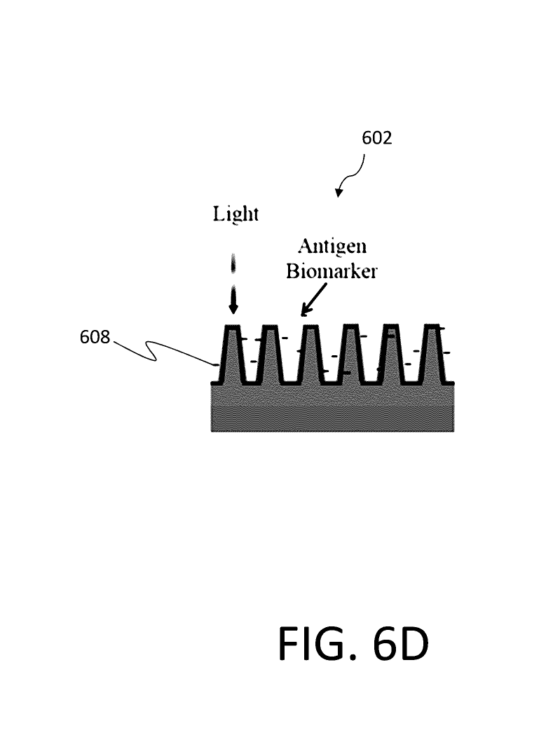

FIG. 6A illustrates one example of a silicon nanowire array 602 incorporated within a test chip 600 that may be used as an optoelectronic sensor system, similar to nanowire solar cells according to certain examples. By shining a light that is absorbed by the nanowire array 602 and sensing change in the electrical signal with illumination, one can determine the concentration of the biomarkers attached to the nanowires within the array 602 since they affect the surface defects on the silicon nanowire surface. FIG. 6B is a further illustration of the nanowire array 602 illustrated in the system of FIG. 6A. As discussed, in certain examples the nanowire array 602 may be functionalized to detect a desired biomarker. FIG. 6B illustrates one such example, where the nanowire array 602 is measured as amine groups 604. In particular embodiments, the nanowire array may further have an EDC-NHS surface modification 606, as illustrated in FIG. 6C. FIG. 6D is an illustration of antigen biomarkers 608 introduced into the nanowire array 602, as performed during operation of the associated biosensor.

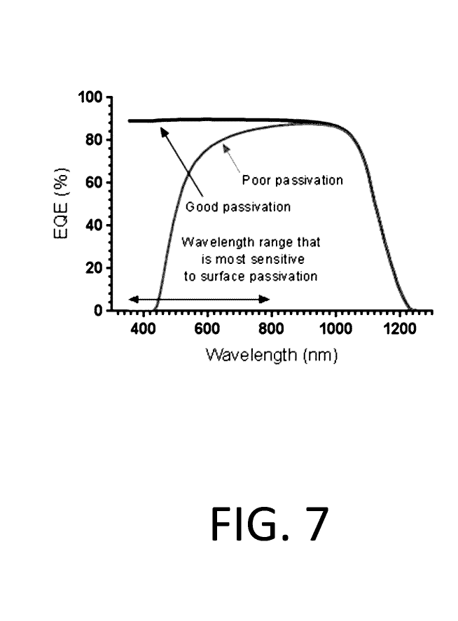

FIG. 7 illustrates a simulated external quantum efficiency (EQE—number of electrons out per photons in) curve for two solar cells. EQE is an optoelectronic measurement during which a sample is exposed to a range of wavelengths. In the illustrated example, the sample was exposed to wavelengths within the range of 350 nm to 1200 nm. The number of electrons generated at each wavelength is subsequently measured. The two solar cells in FIG. 7 are identical except for the front surface passivation quality. For incident light in the wavelength range of 350-700 nm, solar cells are very sensitive to surface passivation.

In certain examples, EQE may be used by the biosensor to detect biomarkers. In some cases, if there are no biomarkers attached to the nanowire array (e.g., nanowire array 602), the biosensor will be unpassivated, and there will be a minimal optoelectronic response. Otherwise, the biosensor will be passivated and the photo-created carriers will be collected from the biosensor leading to a strong optoelectronic response.

In certain examples, the current-voltage of the test chip can be measured with and without illumination using light with wavelengths between 350 nm and 1000 nm, for example. In this example, without the attachment of the biomarkers a surface of the nanowire array (e.g., nanowire array 602) will not be electrically passivated, and any carriers created at the surface of the test nanowire array will recombine. The reverse embodiment is also possible where without the attachment of the biomarkers the nanowire surface will be electrically passivated and with the biomarkers present they will have an increased amount of free carrier recombination. The performance of a solar cell is very dependent on the quality of the surface passivation. As discussed herein, in various examples the nanowire array may be constructed from silicon. Accordingly, references herein to nanowire array may also refer to a silicon nanowire array.

In particular, nanowire solar cells are especially dependent on the quality of surface passivation since they have a high surface area. According to various examples, the biosensor may utilize the sensitivity of electrical properties of the nanowire array (when implemented as a solar cell), in particular, the quality of the front surface passivation, by measuring the solar cell response in a wavelength range that is sensitive to the front surface, for example light with wavelengths between 350 and 700 nm.

Furthermore, in certain examples different sections (e.g., groups of individual nanowire arrays) of the silicon nanowire array can be functionalized to be sensitive to different biomarkers. These subarrays can be electrically isolated using, for example, different techniques to create electrical isolation on a silicon chip (e.g., processes used during microelectronic device fabrication). For example, silicon dioxide trenches can be made to create electrically isolated sections within the associated test chip. These sections of the array can be electrically addressed individually, similar to an array used for memory or displays. In this way, multiple biomarkers can be detected using the same test chip.

The incident light can either be scanned over a desired section of the nanowire array, or “flashed” to illuminate the entire sample. Once illuminated, the current-voltage of each subarray is taken individually, and a measurement for each subarray group is provided.

In addition to electrical detection, the large area of dense nanowires in the various example test chips for biosensors discussed herein makes measuring the change in optical properties easier to measure. Since the optical response of the nanowire arrays also changes due to the binding of cancer biomarkers, the changes in the optical absorption, reflection, luminenscence, or other optical properties can be used to measure the presence of biomarkers within a tested sample. Using these optical measurements along with the current-voltage, quantum efficiency, conductance-voltage or other electrical characteristics of the nanowire arrays, the biosensor can optically and electrically detect cancer biomarkers without electrically contacting both sides (e.g., a top surface and a bottom surface) of the nanowires, as is required by typical horizontal arrangements.

In addition to the various other benefits discussed herein, the redundancy in the nanowire arrays of various examples helps reduce the concerns of false positives because of the two simultaneous measurements (e.g., electrical and optical) for the same biomarker on the same test chip. Accordingly, concerns about false-positives from one measurement of cancer biomarker binding can be confirmed or rejected by having a second independent measurement.

The high sensitivity of the biosensor discussed herein may enable real time detection such that, for example, the biosensor may continuously monitor critically ill patients to study chemotherapy drug level in their blood, optimizing therapeutic benefit and reducing toxicity.

According to various aspects and embodiments, the nanowires can be functionalized, for example, with aminopropyl functional groups/amine groups, and then with antibodies to prostate-specific antigen (PSA) using standard EDC/NHS chemistry. This functionalization gives the nanowires a surface that can bind specifically to PSA antigens. Functionalizing the nanowires can be performed according to various known methods, as will be understood to one of ordinary skill in the art.

One example of a procedure functionalizing the nanowires of various examples, may include incubating the silicon nanowires in a solvent containing (3-Aminopropyl) triethoxysilane (APTES) for a predetermine duration, followed by multiple rounds of washing the material with a solvent. Then, using EDC/NHS chemistry, the PSA-specific antibody may be immobilized on the surface of the array. This functionalization gives the nanowires a charged surface when a desired biomarker is present and a minimal charge when the biomarker is absent.

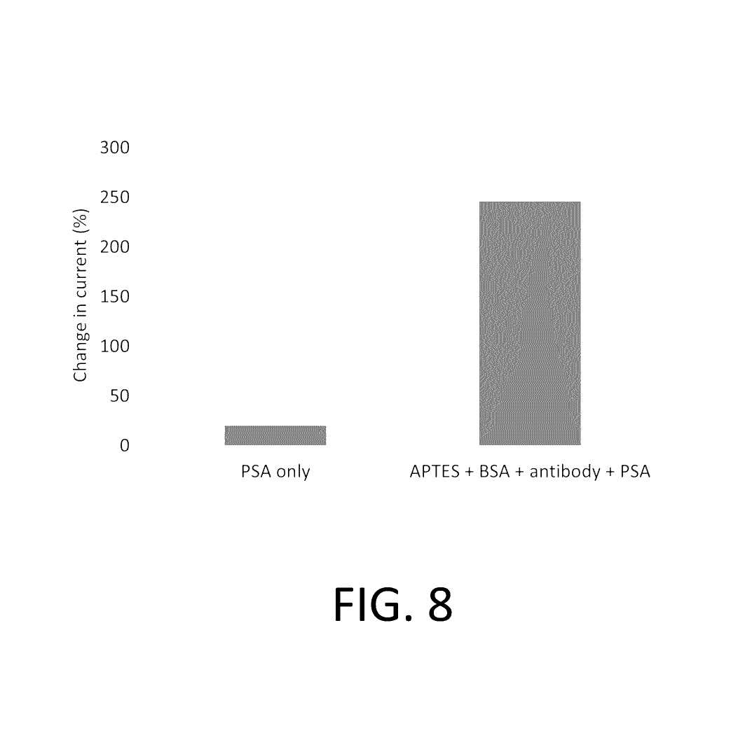

FIG. 8 is a graph illustrating one example of the performance of the nanowires of the array once functionalized as discussed herein. In particular, FIG. 8 shows the percent change in the optically induced current in the nanowire array after exposure to PSA, compared to before exposure. The PSA only sample is a control sample with nanowires without the antibody and other functionalizing components, and the APTES+BSA+antibody+PSA sample has all the functionalizing components.

Referring to the graph of FIG. 8, the current was measured without PSA for both cases and then measured once the PSA was incubated on the surface of the nanowire array. According to various examples, the current is produced by the associated test chips because both samples have an electrical junction (e.g., p-n junction), and thus form a solar cell which responds to incident light with an electrical current. The change in the current before and after PSA exposure is around 20% for the silicon nanowire control sample, as compared to a 250% change for the sample that had all the functionalizing components and PSA. According to various examples, detection of PSA concentration levels down to 10 ng/ml may be measured with the silicon nanowires of various examples.

Existing research has suggested that PSA has high absorption in the 600 nm to 700 nm wavelength range. Accordingly, in certain examples the optical absorption within this wavelength range can be measured to detect biomarkers, in addition to the electrical performance of the test chip (e.g., solar cell). The optical absorption maybe measured using the light reflected from the nanowires as a function of wavelength over a wide range of wavelengths. Kramer Kronig relations may then be used to deduce the optical absorption coefficient in the wavelength range of interest, e.g., 600-700 nm.

Although the established biomarker PSA is provided as one example, the biosensor of various other aspects and embodiments may detect many other types of cancer indicators and health conditions. Specifically, techniques discussed herein may be used to measure specific DNA or RNA mutations, as well as proteins. Some examples of other biomarkers may include AFP (Liver Cancer), BCR-ABL (Chronic Myeloid Leukemia), BRCA1/BRCA2 (Breast/Ovarian Cancer), BRAF V600E (Melanoma/Colorectal Cancer), CA-125 (Ovarian Cancer), CA19.9 (Pancreatic Cancer), CEA (Colorectal Cancer), EGFR (Non-small-cell lung carcinoma), HER-2 (Breast Cancer), KIT (Gastrointestinal stromal tumor), and S100 (Melanoma), among many others.

FIG. 9 is a schematic diagram of one example of a test chip 900. As illustrated, a nanowire array 902 is electrically connected to a substrate 904. A front surface of the nanowire array, as discussed above, is indicated by the directional indicator 906. According to various examples, the nanowire array 902 may include a plurality of individual nanowires. Each individual nanowire is defined by a longitudinal surface and a vertical surface. In certain examples, the longitudinal surface of each nanowire is at least two times longer than the vertical surface, as illustrated in FIG. 9. An axis of each nanowire (e.g., represented by axis 908) extends in a direction substantially perpendicular to the substrate 904 (e.g., in a vertical direction). Alternatively, in certain other examples, each nanowire can be arranged at a substantially non-perpendicular angle relative to the substrate 904 while still extending in the vertical direction. That is, each nanowire may be affixed at any suitable non-horizontal arrangement, according to certain examples. For example, each nanowire may be affixed at an angle of 25, 50 or 85 degrees relative to the substrate 904, where 0 degrees represents a horizontal arrangement on the surface and 90 degrees represents a perpendicular arrangement. As further illustrated in FIG. 9, each nanowire may have by a base-end 914 and a top-end 916, where the vertical surface of each nanowire couples the respective nanowire to the substrate 904 at the base-end 914.

As further illustrated in FIG. 9, the vertical surface of each of the individual nanowires is coupled to the substrate 904. As further discussed above, each individual nanowire may be formed from silicon, and may be coated in a desired chemical material. In certain embodiments, the nanowire array 902 may be split into one or more subarrays (e.g., subarrays 910 and 912), each subarray being coated with a different chemical. Each chemical may be sensitive to a desired biomarker, and may include a biomarker binding agent.

FIG. 10 is an enhanced view of the example test chip 900 illustrated in FIG. 9. As illustrated, the substrate 904 may be composed of a first type of doping 1002 and a second type of doping 1004. Accordingly, the substrate 904 may include a p-n junction 1006 interposed between the first type of doping 1002 and the second type of doping 1004 and formed within the substrate 904. In the illustration of FIG. 10, a base of the nanowire array 1008 is coupled to the substrate 904 at the first type of doping 1002.

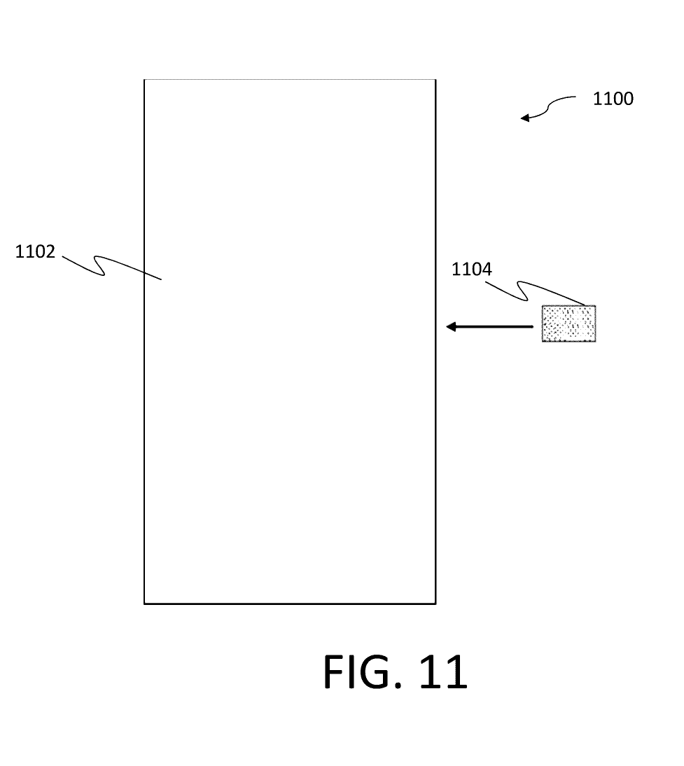

FIG. 11 is a schematic of a sensing system 1100 which includes a measurement tool 1102 and a test chip 1104. As illustrated, the test chip 1104 may be inserted into the measurement tool 1102. In certain examples, the measurement tool 1102 may include the biosensor discussed above, and the test chip 1104 may include the test chip 900 illustrated at least in FIG. 9. As discussed with reference to the various implementations of the biosensor discussed above, the tool 1102 may measure electrical, opto-electrical, and/or optical characteristics of the test chip 1104 when exposed to a sample, such as a PSA containing sample.

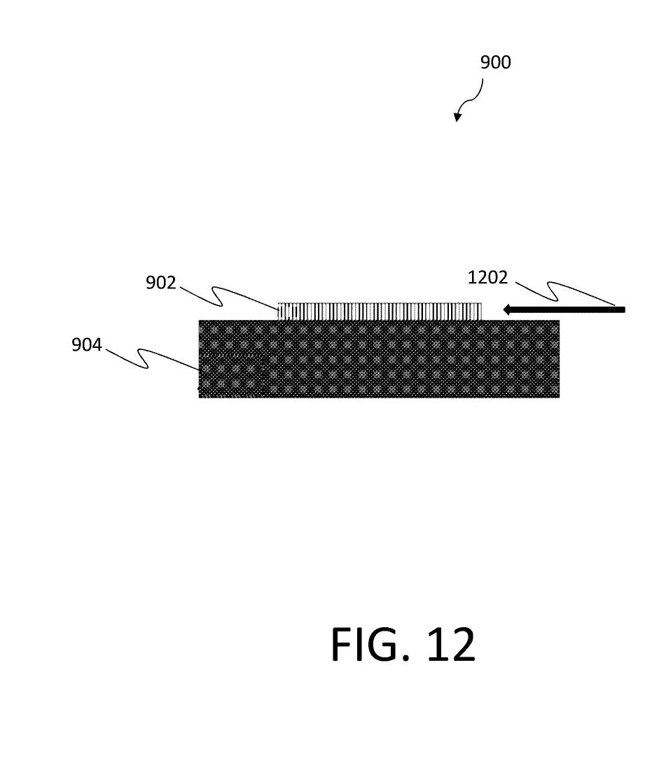

FIG. 12 is another schematic illustration of the example test chip 900 shown in FIG. 9, according to certain implementations. In particular, FIG. 12 illustrates a test sample 1202 being applied (e.g., flowing through) the nanowire array 902 of the test chip 900. Such processes may be applied to detect one or more biomarkers, as discussed herein.

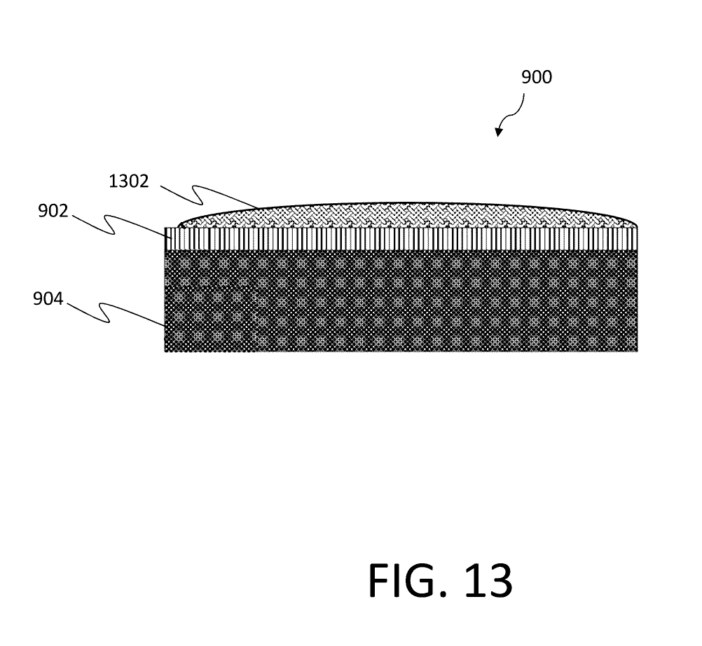

FIG. 13 is another schematic illustration of the example test chip 900 shown in FIG. 9, according to certain aspects. In particular, FIG. 13 illustrates a test sample 1302 being applied to the top surface of the nanowire array 900, as may be performed according to various processes discussed herein.

In various other embodiments, the biosensors discussed herein may be expanded to applications outside medical fields. Such biosensors could include sensors to support the Internet of Things, pollution monitoring, or ensuring high water quality (e.g., detection of undesired bacteria within a water sample). In certain examples, the various implementations of the bionsensors may be portable to provide field detection capability. For instance, the portable biosensors may be configured to detect biological weapons or chemical based weapons, such as anthrax, ricin, and polio, among others.

During operation of the biosensor, the sample can be introduced via a portal. The biosensor can be operated in batch or in continuous mode. When operating in continuous mode, a buffer can be used to wash out earlier samples.

Accordingly, the disclosed aspects and embodiments have an enormous potential across various applications as a result of the ultra-sensitivity, selectivity, ability to measure the number of biomarkers quantitatively, the lack of labels, and real-time detection capabilities discussed herein. In certain applications, the discussed biosensors may reduce the cost of cancer detection when compared to typical imaging techniques such as CT scans, as well as avoid the usual undesirable effects associated therewith, such as exposure to radiation. These approaches will enable early may facilitate early cancer detection and the reduction of cancer patient mortalities.

All patents, patent applications, and publications mentioned in this application are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A nanowire array comprising:
at least 1000 nanowires per cm², wherein the at least 1000 nanowires per cm² include individual nanowires each defined by a longitudinal surface and a vertical surface, the longitudinal surface being at least two times longer than the vertical surface, and wherein the vertical surfaces of each of the individual nanowires is configured to couple to a substrate.

2. The nanowire array according to claim 1, further comprising the substrate wherein each of the individual nanowires is electrically coupled to the substrate.

3. The nanowire array according to claim 2, wherein each of the individual nanowires is further defined by a base-end and a top-end, wherein the base-end of each of the individual nanowires is electrically coupled to the substrate.

4. The nanowire array according to claim 3, wherein each of the individual nanowires is made of silicon.

5. The nanowire array according to claim 3, wherein the substrate further includes at least one p-n junction formed within the substrate.

6. The nanowire array according to claim 2, wherein at least a first nanowire of the individual nanowires is coated with a first chemical material, the first chemical material being sensitive to a first biomarker.

7. The nanowire array according to claim 6, wherein at least a second nanowire of the individual nanowires is coated with a second chemical material, the second chemical material being sensitive to a second biomarker, wherein the second biomarker is distinct from the first biomarker.

8. The nanowire array according to claim 6, wherein a first group of the individual nanowires is coated with a first binding agent, and wherein a second group of the individual nanowires is coated with a second binding agent, the first binding agent and the second binding agent being distinct from each other.

9. The nanowire array according to claim 8, wherein the first group of the individual nanowires is sensitive to a first biomarker, and wherein the second group of the individual nanowires is sensitive to a second biomarker.

10. The nanowire array according to claim 9, wherein the first group of the individual nanowires is electrically isolated from the second group of the individual nanowires.

11. The nanowire array according to claim 2, wherein each of the individual nanowires is configured to provide an electrical measurement.

12. The nanowire array according to claim 11, wherein the electrical measurement includes a current-voltage measurement, and wherein the individual nanowires are further configured to provide the current-voltage measurement in response to receiving incident light.

13. The nanowire array according to claim 12, wherein each of the individual nanowires is configured to receive monochromatic light as the incident light.

14. The nanowire array according to claim 13, wherein a wavelength of the monochromatic light includes a range of scanned wavelengths.

15. The nanowire array according to claim 14, wherein the range of scanned wavelengths is substantially within the range of 350 nm to 700 nm.

16. A nanowire-based biosensor comprising:
a substrate; and
an array of nanowires including a plurality of individual nanowires arranged at a density of least 1000 nanowires per cm², each individual nanowire of the plurality being defined by a longitudinal surface and a vertical surface and being coupled to the substrate,
wherein each of the plurality of individual nanowires is configured to be operable to detect a biomarker optically and electrically.

17. The nanowire-based biosensor according to claim 16, wherein each of the plurality of nanowires is coated with a chemical sensitive to the biomarker.

18. The nanowire-based biosensor according to claim 16, wherein in detecting the biomarker electrically, each of the plurality of individual nanowires is operable to detect the biomarker based on quantum efficiency.

19. The nanowire-based biosensor according to claim 15, wherein each of the individual nanowires is operable to detect the biomarker at a wavelength range of substantially 350 nm to 700 nm.

20. A cancer monitoring method comprising:
providing a sample to a nanowire array including at least 1000 nanowires per cm², wherein the at least 1000 nanowires per cm² include individual nanowires each defined by a longitudinal surface and a vertical surface, the longitudinal surface being at least two times longer than the vertical surface;
illuminating the nanowire array with light within a predetermine range of wavelengths; and
measuring an electrical response of the nanowire array to determine the presence of one or more biomarkers within the sample.
